(19) United States
(12) Patent Application Publication
(10) Pub. No.: US 2015/0056195 A1
(43) Pub. Date: Feb. 26, 2015

(54) COMPOSITIONS AND METHODS FOR INIHIBITING TUMORIGENICITY OF SENESCENT CANCER CELLS INDUCED BY CHEMOTHERAPY

(71) Applicant: INSERM (INSTITUT NATIONAL DE LA SANTE ET DE LA RECHERCHE MEDICALE), Paris (FR)

(72) Inventor: Corine BERTOLOTTO-BALLOTTI, NICE (FR)

(21) Appl. No.: 14/465,926

(22) Filed: Aug. 22, 2014

Related U.S. Application Data
(60) Provisional application No. 61/869,103, filed on Aug. 23, 2013.

(51) Int. Cl. C07K 16/28; A61K 31/437; A61K 39/395; A61K 31/506; A61K 45/06; C07K 16/24

(52) U.S. Cl. CPC C07K 16/2866; A61K 45/06; C07K 16/248; A61K 39/3955; A61K 31/506; A61K 31/437; C07K 2317/24
USPC 424/133.1; 424/142.1

(57) ABSTRACT

The invention relates to the field of oncology and to chemotherapy resistance and relapse. Thus the invention provides compositions and methods for inhibiting tumorigenicity of senescent cancer cells induced by a chemotherapeutic agent. The invention also provides compositions and methods for inhibiting conversion of non-stem cancer cells (non-CSCs) into tumor initiating cancer stem cells (CSCs) induced by a chemotherapeutic agent.

### CROSS-REFERENCE TO RELATED APPLICATIONS

This application claims priority to U.S. Provisional Application 61/869,103 filed Aug. 23, 2013, and the complete contents thereof is herein incorporated by reference.

### FIELD OF THE INVENTION

The invention relates to the field of oncology and to chemotherapy resistance and relapse. The invention thus provides compositions and methods for inhibiting tumorigenicity of senescent cancer cells induced by a chemotherapeutic agent. The invention also provides compositions and methods for inhibiting conversion of non-stem cancer cells (non-CSCs) into tumor initiating cancer stem cells (CSCs) induced by a chemotherapeutic agent.

### BACKGROUND OF THE INVENTION

Melanoma cells are notoriously known for their high resistance to almost all therapeutic treatments. It was recently proposed that the remarkable phenotypic plasticity of melanoma cells allows for the rapid development of both resistance to chemotherapeutic drugs and invasive properties^{1, 2}. At a given time, within a tumor or in culture, not all the melanoma cells have the same capacity to form tumors. A small population is endowed with high tumorigenic potential and has been qualified as Melanoma Initiating Cells (MIC), even though, a reversible phenotypic switch exists between these MIC and their less tumorigenic progeny³. Until now no consensual marker, which characterizes the MIC population, has been identified⁴⁻⁸. Nevertheless, the most tumorigenic melanoma cells appear to have a poorly differentiated phenotype, with high expression of mesenchymal markers^{3, 6, 9-11}.

Interestingly, the equilibrium between the low-MITF/slow growing, highly tumorigenic and the high-MITF/fast growing, poorly tumorigenic populations can be modified by external stimuli such as a differentiation signal or oxygen level⁹. Therefore, any stimulus that could change this balance, may alter the tumorigenicity of melanoma.

Meanwhile, we showed that melanoma cells could undergo premature senescence upon MITF depletion or upon chemotherapy treatments¹³⁻¹⁵. Senescence can be associated in some circumstances with the production of a secretome composed of several pro-inflammatory factors^{16, 17}. In senescent melanoma cells, we demonstrated the existence of such a secretome¹⁸. While some reports indicated that components of the secretome associated with senescence could reinforce the senescence program^{19, 20} other studies demonstrated that it displays deleterious effects, favoring migration, invasion and epithelial-to-mesenchymal transition^{21, 22}. In agreement with these results, we found that exposure of naive, non-tumorigenic 501mel melanoma cells with the secretome of senescent melanoma cells (SSMC), rendered them highly tumorigenic¹⁸. These observations raise the possibility that chemotherapy treatments may have deleterious effects by promoting the tumorigenic potential of cryptic, residual melanoma cells, not affected by the treatment, and thus contribute to the recurrence of the disease. Indeed, it has been previously reported that chemotherapy drugs, used in melanoma treatment, entail a senescence-like phenotype in melanoma cells that is associated with the production of SSMC endowed with pro-tumorigenic properties¹⁸.

Very recently, it has been disclosed that Vemurafenib (a B-Raf enzyme inhibitor developed by Plexxikon and Genentech and marketed as Zelboraf for the treatment of late-stage melanoma) induces senescence features in melanoma cells⁴⁹. These observations provide a possible explanation for the lack of complete and durable pro-apoptotic effect of Vemurafenib in patients since senescent cells can provide a microenvironment that increases the metastatic abilities of neighboring cells and might thereby contribute to resistance. Furthermore, cancer cells that were driven into senescence by chemotherapeutic agents can develop chemoresistant side populations with cancer stem cell-like properties and therefore drug-resistant cells with more aggressive tumorigenic phenotypes than before treatment.

Hence, it is highly desirable to understand the involvement of senescence in melanoma regression and resistance in chemotherapy-treated patients (such as in Vemurafenib-treated patients) in order to identify new means for inhibiting tumorigenicity induced by chemotherapeutic agents and thus improving clinical outcomes for said patients.

### SUMMARY OF THE INVENTION

In a first aspect, the invention relates to a product containing (i) a chemotherapeutic agent and (ii) an inhibitor of the signal transducer and activator of transcription 3 (STAT3) signaling pathway, as a combined preparation for simultaneous, separate or sequential for use in a method for inhibiting the tumorigenicity of senescent cancer cells induced by said chemotherapeutic agent.

In a second aspect, the invention relates to a pharmaceutical composition comprising (i) a chemotherapeutic agent and (ii) an inhibitor of STAT3 signaling pathway, for use in a method for inhibiting the tumorigenicity of senescent cancer cells induced by said chemotherapeutic agent.

In a third aspect, the invention relates to a product containing (i) a chemotherapeutic agent and (ii) an inhibitor of STAT3 signaling pathway, as a combined preparation for simultaneous, separate or sequential for use in a method for inhibiting the conversion of non-stem cancer cells (non-CSCs) into cancer stem cells (CSCs) induced by said chemotherapeutic agent.

In a fourth aspect, the invention relates to a pharmaceutical composition comprising (i) a chemotherapeutic agent and (ii) an inhibitor of STAT3 signaling pathway, for use in a method for inhibiting the conversion of non-CSCs into CSCs induced by said chemotherapeutic agent.

In a fifth aspect, the invention relates to a pharmaceutical composition or a kit-of-part composition comprising an anti-IL-6 antibody and a V600E B-Raf inhibitor.

In a sixth aspect, the invention relates to a pharmaceutical composition or a kit-of-part composition comprising an anti-IL-6R antibody and a V600E B-Raf inhibitor.

### DETAILED DESCRIPTION OF THE INVENTION

The invention relies on the fact that the inventors have now shown how the secretome of melanoma cells entering senescence upon chemotherapy treatments increases the tumorigenicity of naïve melanoma cells by inducing, through STAT3 activation, a melanoma-initiating cell phenotype that could favor chemotherapy resistance and relapse. They have shown that secretome of senescent melanoma cells (SSMC) drives naïve melanoma cells towards a more mesenchymal and stem-like phenotype. SSMC also favor melanoma tumorigenicity by increasing MIC population. They have especially found that the SSMC activates the STAT3 pathway and STAT3 inhibition prevents secretome effects, including the acquisition of tumorigenic properties. It results that they have pointed out, both in vitro and in vivo, the key role of the STAT3 signaling cascade in acquisition of the stemness and mesenchymal phenotype as well as the melanoma-initiating properties mediated by SSMC. Targeting the STAT3 pathway might help to circumvent the detrimental effects of the SSMC and to strengthen the efficiency of anti-melanoma therapies.

Without wishing to be bound by theory, the inventors have discovered that said a secretome is produced by senescent cancer cells in response to the chemotherapeutic agent, thus stimulating tumorigenicity, proliferation and migration of the remaining non-injured cancer cells as well as favoring their reprogramming into tumor-initialing cancer stem cells. This phenomenon would finally lead to maintain niches of cancer cells such as melanoma cells that contribute to cancer relapses.

### Therapeutic Methods and Uses

Accordingly, in a first aspect of the invention relates to a product containing (i) at least a chemotherapeutic agent and (ii) at least an inhibitor of the signal transducer and activator of transcription 3 (STAT3) signaling pathway, as a combined preparation for simultaneous, separate or sequential for use in a method for inhibiting tumorigenicity of senescent cancer cells induced by said chemotherapeutic agent.

The invention also relates to a pharmaceutical composition comprising (i) at least a chemotherapeutic agent and (ii) at least an inhibitor of STAT3 signaling pathway, for use in a method for inhibiting tumorigenicity of senescent cancer cells induced by said chemotherapeutic agent.

As used herein, the term “tumorigenicity” refers to the functional features of a solid tumor stem cell including the properties of self-renewal (giving rise to additional tumorigenic cancer stem cells) and proliferation to generate all other tumor cells (giving rise to differentiated and thus non-tumorigenic tumor cells) that allow solid tumor stem cells to form a tumor. These properties of self-renewal and proliferation to generate all other tumor cells confer on cancer stem cells the ability to form tumors upon serial transplantation into an immunocompromised host (e.g., a mouse) compared to non-tumorigenic tumor cells, which are unable to form tumors upon serial transplantation. In the case of melanoma, as described herein there is a plasticity allowing the conversion of non-tumorigenic melanoma cells towards quiescent initiating melanoma cells. It has been observed that non-tumorigenic tumor cells may form a tumor upon primary transplantation into an immunocompromised host after obtaining the tumor cells from a solid tumor, but those non-tumorigenic tumor cells do not give rise to a tumor upon serial transplantation.

As used herein, the terms “cancer cell” or “tumor cell” are used interchangeably and refer to the total population of cells derived from a tumor or a pre-cancerous lesion, including both non-tumorigenic cells, which comprise the bulk of the tumor cell population, and tumorigenic stem cells (cancer stem cells).

As used herein, the term “senescent cancer cells” refers cancer cells undergoing in senescence in response to chemotherapy. Senescent cancer cells are non-growing and possess one or more of the following additional characteristics attributed to senescent cells: heterochromatin formation, changes in cell shape, increased senescence-associated β-galactosidase activity. Moreover, senescent cancer cells produce a secretome comprising numerous cytokines including CCL2 as previously disclosed in Ohanna et al., 2011.

Cancer cells susceptible to become senescent cancer cells are selected among melanoma, breast cancer, prostate cancer, liver cancer, bladder cancer, lung cancer, colon cancer, colorectal cancer, gastrointestinal cancer, pancreatic cancer, cervical cancer, ovarian cancer, bladder cancer, kidney cancer and various types of head and neck cancer.

In a particular embodiment, the senescent cancer cells are senescent melanoma cells.

As used herein, the term “chemotherapeutic agent” refers to compounds which are used in the treatment of cancer and that have the functional property of inhibiting a development or progression of a neoplasm in a human, particularly a malignant (cancerous) lesion, such as a carcinoma, sarcoma, lymphoma, or leukemia.

Chemotherapeutic agents have different modes of actions, for example, by influencing either DNA or RNA and interfering with cell cycle replication. Examples of chemotherapeutic agents that act at the DNA level or on the RNA level are anti-metabolites (such as Azathioprine, Cytarabine, Fludarabine phosphate, Fludarabine, Gemcitabine, cytarabine, Cladribine, capecitabine 6-mercaptopurine, 6-thioguanine, methotrexate, 5-fluoroouracil and hyroxyurea; alkylating agents (such as Melphalan, Busulfan, Cis-platin, Carboplatin, Cyclophosphamide, Ifosphamide, Dacarabazine, Fotemustine, Procarbazine, Chlorambucil, Thiotepa, Lomustine, Temozolomide); anti-mitotic agents (such as Vinorelbine, Vincristine, Vinblastine, Docetaxel, Paclitaxel); topoisomerase inhibitors (such as Doxorubincin, Amsacrine, Irinotecan, Daunorubicin, Epirubicin, Mitomycin, Mitoxantrone, Idarubicin, Teniposide, Etoposide, Topotecan); antibiotics (such as actinomycin and bleomycin); asparaginase; anthracyclines or taxanes.

Other chemotherapeutic agents are tyrosine kinase inhibitors (TKI). A number of other TKIs are in late and early stage development for treatment of various types of cancer. Examplary TKIs include, but are not limited to: BAY 43-9006 (Sorafenib, Nexavar®) and SU11248 (Sunitinib, Sutent®), Imatinib mesylate (Gleevec®, Novartis); Gefitinib (Iressa®, AstraZeneca); Erlotinib hydrochloride (Tarceva®, Genentech); Vandetanib (Zactima®, AstraZeneca), Tipifarnib (Zarnestra®, Janssen-Cilag); Dasatinib (Sprycel®, Bristol Myers Squibb); Lonafarnib (Sarasar®, Schering Plough); Vatalanib succinate (Novartis, Schering AG); Lapatinib (Tykerb®, GlaxoSmithKline); Nilotinib (Novartis); Lestaurtinib (Cephalon); Pazopanib hydrochloride (GlaxoSmithKline); Axitinib (Pfizer); Canertinib dihydrochloride (Pfizer); Pelitinib (National Cancer Institute, Wyeth); Tandutinib (Millennium); Bosutinib (Wyeth); Semaxanib (Sugen, Taiho); AZD-2171 (AstraZeneca); VX-680 (Merck, Vertex); EXEL-0999 (Exelixis); ARRY-142886 (Array BioPharma, AstraZeneca); PD-0325901 (Pfizer); AMG-706 (Amgen); BIBF-1120 (Boehringer Ingelheim); SU-6668 (Taiho); CP-547632 (OSI); (AEE-788 (Novartis); BMS-582664 (Bristol-Myers Squibb); JNK-401 (Celgene); R-788 (Rigel); AZD-1152 HQPA (AstraZeneca); NM-3 (Genzyme Oncology); CP-868596 (Pfizer); BMS-599626 (Bristol-Myers Squibb); PTC-299 (PTC Therapeutics); ABT-869 (Abbott); EXEL-2880 (Exelixis); AG-024322 (Pfizer); XL-820 (Exelixis); OSI-930 (OSI); XL-184 (Exelixis); KRN-951 (Kirin Brewery); CP-724714 (OSI); E-7080 (Eisai); HKI-272 (Wyeth); CHIR-258 (Chiron); ZK-304709 (Schering AG); EXEL-7647 (Exelixis); BAY-57-9352 (Bayer); BIBW-2992 (Boehringer Ingelheim); AV-412 (AVEO); YN-968D1 (Advenchen Laboratories); Staurosporin, Midostaurin (PKC412, Novartis); Perifosine (AEterna Zentaris, Keryx, National Cancer Institute); AG-024322 (Pfizer); AZD-1152 (AstraZeneca); ON-01910Na (Onconova); and AZD-0530 (AstraZeneca).

Additional chemotherapeutic agents may be selected from, but are not limited to, cytokines, chemokines, growth factors, growth inhibitory factors, hormones, soluble receptors, decoy receptors, monoclonal or polyclonal antibodies, mono-specific, bi-specific or multi-specific antibodies, monobodies, polybodies.

Exemplary biologics drugs include, but are not limited to: anti-angiogenic agents such as Bevacuzimab (mAb, inhibiting VEGF-A, Genentech); IMC-1121B (mAb, inhibiting VEGFR-2, ImClone Systems); CDP-791 (Pegylated DiFab, VEGFR-2, Celltech); 2C3 (mAb, VEGF-A, Peregrine Pharmaceuticals); VEGF-trap (soluble hybrid receptor VEGF-A, PIGF (placenta growth factor) Aventis/Regeneron).

In one particular embodiment, the chemotherapeutic agent is an anti-melanoma drug selected from the group consisting of Dabrafenib, Dacarbazine, Fotemustine, Iipilimumab, Selumetinib, Temozolomide, Trametinib and Vemurafenib.

In another embodiment, the chemotherapeutic agent is a selective B-Raf inhibitor, preferably a V600E B-Raf inhibitor such as PLX-4032 (also referred to as Vemurafenib marketed by Roche and Plexxikon and described in U.S. Pat. No. 7,863,288), PLX-4720 GSK2118436 (also referred to as Dabrafenib, marketed by GlaxoSmithKline and described in U.S. Pat. No. 7,994,185), GDC-0879, XL281 (BMS-908662) and AZ628. B-Raf inhibitors are well known in the art and are described for instance in Zambon A; BRAF as a therapeutic target: a patent review (2006-2012); Expert Opin Ther Pat. 2013 February; 23(2):155-64.

In one embodiment, the inhibitor of STAT3 signaling pathway is a STAT3 inhibitor.

As used herein, the term “Signal transducer and activator of transcription 3” (STAT3) is well known in the art and refers to a member of a family of DNA-binding proteins that plays an important role in cytokine signal transduction. Phosphorylation on Tyr705 results in its activation Two phosphorylated and activated STAT3 monomers dimerize through reciprocal pTyr-SH2 domain interactions, translocate to the nucleus, and bind to specific DNA-response elements of target genes, thereby inducing gene transcription. STAT3 has been found to be responsive to interleukin-6 (IL-6). The STAT3 gene encodes a 770 amino acid polypeptide and the naturally occurring human STAT3 protein has an aminoacid sequence as shown in Uniprot Accession number NP_{—}644805.

As used herein, the term “STAT3 inhibitor” refers to a molecule (natural or synthetic) which inhibit signalling through STAT3, as well as compounds which inhibit the expression of the STAT3 gene. They include compounds which inhibit the activity of STAT3, or by inhibiting STAT3 signalling by other mechanisms. STAT3 inhibitors are well known in the art and are described for instance in Page B D et al., Signal transducer and activator of transcription 3 inhibitors: a patent review. Expert Opin Ther Pat. 2011 January; 21(1):65-83.

Typically, STAT3 inhibitor are WP1066, SPI (a 28-mer peptide, derived from the STAT3 SH2 domain) disclosed in WO 2011/163423, substituted purine analogs (e.g. substituted 2-(9H-purin-9-yl) acetic acid analogues) disclosed in WO 2011/163424, and N-[1,3,4-oxadiazol-2-yl]-4-quinolinecarboxamide derivatives disclosed in WO 2010/004761.

In one embodiment, the STAT3 inhibitor is an inhibitor of STAT3 gene expression.

An “inhibitor of expression” refers to a natural or synthetic compound that has a biological effect to inhibit the expression of a gene.

Therefore, an “inhibitor of STAT3 gene expression” denotes a natural or synthetic compound that has a biological effect to inhibit the expression of STAT3 gene.

In a particular embodiment, the inhibitor of STAT3 gene expression is selected form the group consisting of an antisense oligonucleotide, a small inhibitory RNA (siRNA) and a ribozyme. Accordingly, several approaches for inhibiting STAT3 expression have been disclosed in U.S. Pat. Nos. 5,719,042 and 5,844,082 and in International Patent applications Nos. WO 00/61602, WO 2005/083124 and WO 2012/161806.

In another embodiment, the inhibitor of STAT3 signaling pathway is an IL-6 antagonist.

As used herein, the term “IL-6 antagonist” refers to a molecule (natural or synthetic) that blocks signal transduction by IL-6 and inhibit the biological activity of IL-6. Specific examples of IL-6 antagonists include molecules that bind to IL-6, molecules that inhibit IL-6 expression, molecules that bind to an IL-6 receptor, molecules that inhibit the expression of an IL-6 receptor, molecules that bind to gpl30, and molecules that inhibit gpl30 expression. Preferably, IL-6 antagonists include anti-IL-6 antibodies, anti-IL-6 receptor antibodies, anti-gpl30 antibodies, soluble IL-6 receptors, partial peptides of IL-6 or IL-6 receptor, as well as low-molecular-weight compounds, antisense or siRNAs directed to IL-6 or IL-6R.

As used herein, the term “Interleukin-6” (IL-6) refers to a protein involved in the regulation of immune responses such as lymphocyte differentiation. IL-6 is well known in the art and is also known as B cell-stimulating factor 2 (BSF-2) or interferon beta-2 (IFNB2). The IL-6 gene encodes a 212 amino acid polypeptide and the naturally occurring human IL-6 protein has an aminoacid sequence as shown in Uniprot Accession number P05231. Naturally occurring human IL-6 variants are known and included in this definition.

As used herein, the term “anti-interleukin-6 antibody” (anti-IL-6 antibody) refers to an antibody to IL-6 where the antibody binds to IL-6 and antagonizes (i.e., inhibits or neutralizes) IL-6 activity.

An example of such an antibody is sirukumab (CNTO 136) a human monoclonal antibody designed for the treatment of rheumatoid arthritis currently under development by Johnson & Johnson's subsidiary Centocor.

Another example of anti-IL-6 antibody is siltuximab (CNTO 328) a chimeric monoclonal antibody designed for the treatment of cancer currently developed by Janssen.

Patent publications related to anti-IL-6R antibodies include WO 2004/039826; WO 2004/045507; WO 2006/119115; WO 2007/076927 and WO 2008/019061.

In one particular embodiment, the anti-IL-6R antibody is sirukumab or siltuximab

As used herein, the term “interleukin-6 receptor” (IL-6R) refers to a type I cytokine receptor. IL-6R is well known in the art and refers to a protein complex consisting of an IL-6 receptor subunit (also known as CD126) and IL-6 signal transducer Glycoprotein 130 (also known as CD130). IL-6 binds to the IL-6 receptor subunit and initiates the association of IL-6 receptor subunit and gpl30 resulting in a high affinity receptor and signal transduction leading to, e.g. phosphorylation of signal transducer and activator of transcription 3 (STAT3).

Interleukin-6 is an inflammatory cytokine, which activates STAT3 through binding to one IL-6 receptor and two gpl 30 molecules. Then STAT3 molecules are recruited to gpl30 and are phosphorylated by Janus kinases (JAK). Phosphorylated STAT3 molecules dimerize via Src homology-2 (SH2) domain and translocate to the nucleus, where the STAT3 dimer binds to a specific DNA element to regulate downstream genes, which are involved in cell proliferation, angiogenesis, and anti-apoptosis.

As used herein, the term “anti-interleukin-6 receptor antibody” (anti-IL-6R antibody) refers to an antibody to IL-6 receptor where the antibody binds to IL-6 receptor and antagonizes (i.e., inhibits or neutralizes) IL-6 receptor activity.

An example of such an antibody is tocilizumab, a humanized IL-6R monoclonal antibody developed by Hoffmann-La Roche and Chugai under the trade names Actemra and RoActemra) (see, e.g., Sato et al., Cancer Res 1993; 53: 851-6; and U.S. Pat. No. 7,479,543) that is used for the treatment of rheumatoid arthritis.

Another example of anti-IL-6R antibody is sarilumab currently co-developed Regeneron and Sanofi (see, e.g., U.S. Pat. No. 7,582,298).

Patents and patent publications related to anti-IL-6R antibodies include: U.S. Pat. No. 5,171,840, U.S. Pat. No. 5,480,796, U.S. Pat. No. 5,670,373, U.S. Pat. No. 5,851,793, U.S. Pat. No. 5,795,965, U.S. Pat. No. 5,817,790, U.S. Pat. No. 7,479,543, U.S. Pat. No. 5,888,510, U.S. Pat. No. 6,086,874, U.S. Pat. No. 6,261,560, U.S. Pat. No. 6,692,742, U.S. Pat. No. 7,566,453, U.S. Pat. No. 7,824,674, U.S. Pat. No. 7,320,792, U.S. Pat. No. 7,955,598, WO 2007/143168, U.S. Pat. No. 7,582,298 and WO 2011/085158.

In one particular embodiment, the anti-IL-6R antibody is tocilizumab or sarilumab.

Alternatively, in another embodiment the IL-6 antagonist is small compound useful in inhibiting IL6-mediated STAT3 phosphorylation such as substituted 2-(1H-indol-3-yl)ethanol analogs described in international patent application No. WO2013/019690.

In a second aspect of the invention relates to a product containing (i) at least a chemotherapeutic agent and (ii) at least an inhibitor of STAT3 signaling pathway, as a combined preparation for simultaneous, separate or sequential for use in a method for inhibiting the conversion of non-stem cancer cells (non-CSCs) into cancer stem cells (CSCs) induced by said chemotherapeutic agent.

The invention also relates to a pharmaceutical composition comprising (i) at least a chemotherapeutic agent and (ii) at least an inhibitor of STAT3 signaling pathway, for use in a method for inhibiting the conversion of non-CSCs into CSCs induced by said chemotherapeutic agent.

As used herein, the terms “cancer cell” or “tumor cell” are used interchangeably and refer to the total population of cells derived from a tumor or a pre-cancerous lesion, including both non-tumorigenic cells, which comprise the bulk of the tumor cell population, and tumorigenic stem cells (cancer stem cells).

As used herein, the terms “non-tumorigenic”, “naive”, “non-cancer stem cell” or “non-CSC” are used interchangeably and refer to those tumor cells lacking the capacity to renew and differentiate to distinguish those tumor cells from cancer stem cells.

As used herein, the terms “cancer stem cell”, “CSC”, “tumor stem cell”, “tumor initiating cell” are used interchangeably herein and refer to a population of cells from a solid tumor that: (1) have extended proliferative capacity; 2) are capable of asymmetric cell division to generate one or more kinds of differentiated progeny with proliferative or developmental potential; and (3) are capable of symmetric cell divisions for self-renewal or self-maintenance. These properties confer on the “cancer stem cells” or “tumor initiating cells” the ability to form tumors upon serial transplantation into an immunocompromised host (e.g., a mouse) compared to the majority of tumor cells that fail to form tumors. Cancer stem cells undergo self-renewal versus differentiation in a manner to form tumors with abnormal cell types that can change over time as mutations occur. Accordingly, melanoma initiating cells display an increased expression of mesenchymal and stemness markers as described in the section EXAMPLES (below).

As used herein, the term “cancer” refers to the pathological condition in mammals that is typically characterized by unregulated cell growth. Examples of cancer include, but are not limited to, carcinoma, lymphoma, blastoma, sarcoma, and leukemia.

More particular examples of such cancers include melanoma, breast cancer, prostate cancer, liver cancer, bladder cancer, lung cancer, colon cancer, colorectal cancer, gastrointestinal cancer, pancreatic cancer, cervical cancer, ovarian cancer, bladder cancer, kidney cancer and various types of head and neck cancer.

Preferably, the cancer is a melanoma.

As used herein, the term “melanoma” refers to all types of melanoma, including, cutaneous melanoma, extracutaneous melanoma, superficial spreading melanoma, malignant melanoma, nodular melanoma, nodular malignant melanoma, polypoid melanoma, acral lentiginous melanoma, lentiginous malignant melanoma, lentigo maligna melanoma, mucosal lentignous melanoma, mucosal melanoma, soft-tissue melanoma, and desmoplastic melanoma. The term “melanoma” includes primary melanoma and metastatic melanoma.

In a particular embodiment, the non-CSCs are naive melanoma cells and the CSCs are melanoma initiating cells (MICs).

In a third aspect of the invention relates to a product containing (i) at least a chemotherapeutic agent and (ii) at least an inhibitor of STAT3 signaling pathway, as a combined preparation for simultaneous, separate or sequential for use in a method for improving the survival time of a patient treated with said chemotherapeutic agent.

The invention also relates to a pharmaceutical composition comprising (i) at least a chemotherapeutic agent and (ii) at least an inhibitor of STAT3 signaling pathway, in a method for improving the survival time of a patient affected with a cancer and treated with said chemotherapeutic agent.

As used herein, the term “Survival Time” includes “Progression-Free Survival” and “Overall Survival”.

The term “Progression-Free Survival” (PFS) in the context of the invention refers to the length of time during and after treatment during which, according to the assessment of the treating physician or investigator, the patient's disease does not become worse, i.e., does not progress. As the skilled person will appreciate, a patient's progression-free survival is improved or enhanced if the patient experiences a longer length of time during which the disease does not progress as compared to the average or mean progression free survival time of a control group of similarly situated patients.

The term “Overall Survival” (OS) in the context of the invention refers to the average survival of the patient within a patient group. As the skilled person will appreciate, a patient's overall survival is improved or enhanced, if the patient belongs to a subgroup of patients that has a statistically significant longer mean survival time as compared to another subgroup of patients. Improved overall survival may be evident in one or more subgroups of patients but not apparent when the patient population is analysed as a whole.

The invention also relates to a method for inhibiting tumorigenicity of senescent cancer cells induced by said chemotherapeutic agent in a patient in need thereof comprising a step of comprising administering a therapeutically effective amount of an inhibitor of STAT3 signaling pathway to said patient.

The invention also relates to a method for inhibiting the conversion of non-CSCs into CSCs induced by said chemotherapeutic agent in a patient in need thereof comprising a step of comprising administering a therapeutically effective amount of an inhibitor of STAT3 signaling pathway to said patient.

The invention further relates to a method for improving the survival time of a patient in need thereof, said method comprising a step of administering a therapeutically effective amount a step of comprising administering a therapeutically effective amount of an inhibitor of STAT3 signaling pathway to said patient.

By a “therapeutically effective amount” of an inhibitor of STAT3 signaling pathway as above described is meant a sufficient amount of the antagonist to prevent or inhibit a tumorigenicity of senescent cancer cells (e.g. by inhibiting or reducing the deleterious effects caused by the secretome of senescent cancer cells) and thereby prevent or inhibit the conversion of non-CSCs into CSCs. It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

As used herein, the term “patient in need thereof” denotes a mammal, such as a rodent, a feline, a canine, and a primate. Preferably, a patient in need thereof according to the invention is a human. In one embodiment, the patient in need thereof has developed cancer (e.g. is affected with melanoma) and is or will be treated with a chemotherapeutic agent.

### Combination Therapies of the Invention

In other embodiments, the inhibitors of STAT3 signaling pathway may be administered to a patient with an appropriate additional therapeutic agent useful in the treatment of the cancer affecting the patient. The administration of an inhibitor of STAT3 signaling pathway and a chemotherapeutic agent can be carried out simultaneously, e.g., as a single composition or as two or more distinct compositions using the same or different administration routes. Alternatively, or additionally, the administration can be done sequentially, in any order. Alternatively, or additionally, the steps can be performed as a combination of both sequentially and simultaneously, in any order. In certain embodiments, intervals ranging from minutes to days, to weeks to months, can be present between the administrations of the two or more compositions. For example, the chemotherapeutic agent may be administered first, followed by the inhibitor of STAT3 signaling pathway. However, simultaneous administration or administration of the inhibitor of STAT3 signaling pathway first is also contemplated.

Accordingly, the invention further relates to a pharmaceutical composition comprising an inhibitor of STAT3 signaling pathway according to the invention and a chemotherapeutic agent.

In one embodiment, the pharmaceutical composition comprises an anti-interleukin-6 antibody and a chemotherapeutic agent.

In one particular embodiment, the anti-interleukin-6 antibody is sirukumab or siltuximab.

In another particular embodiment, the chemotherapeutic agent is a selective B-Raf inhibitor and preferably a V600E B-Raf inhibitor.

In one preferred embodiment, the V600E B-Raf inhibitor is dabrafenib or vemurafenib.

In a most preferred embodiment, the pharmaceutical composition comprises sirukumab or siltuximab and dabrafenib or vemurafenib.

In another embodiment, the pharmaceutical composition comprises an anti-interleukin-6 receptor antibody and a chemotherapeutic agent.

In one particular embodiment, the anti-interleukin-6 receptor antibody tocilizumab or sarilumab

In another particular embodiment, the chemotherapeutic agent is a selective B-Raf inhibitor and preferably a V600E B-Raf inhibitor.

In a most preferred embodiment, the pharmaceutical composition comprises tocilizumab or sarilumab and dabrafenib or vemurafenib.

In another aspect, the invention relates to a kit-of-part composition comprising an inhibitor of STAT3 signaling pathway according to the invention and a chemotherapeutic agent.

The terms “kit”, “product” or “combined preparation”, as used herein, define especially a “kit-of-part” in the sense that the combination partners as defined above can be dosed independently or by use of different fixed combinations with distinguished amounts of the combination partners, i.e. simultaneously or at different time points. The parts of the kit of parts can then, e.g., be administered simultaneously or chronologically staggered, that is at different time points and with equal or different time intervals for any part of the kit of parts. The ratio of the total amounts of the combination partners to be administered in the combined preparation can be varied. The combination partners can be administered by the same route or by different routes. When the administration is sequential, the first partner may be for instance administered 1, 2, 3, 4, 5, 6, 12, 18 or 24 h before the second partner.

In one embodiment, the kit-of-part composition comprises an anti-interleukin-6 antibody and a chemotherapeutic agent.

In one particular embodiment, the anti-interleukin-6 antibody is sirukumab or siltuximab.

In another particular embodiment, the chemotherapeutic agent is a selective B-Raf inhibitor and preferably a V600E B-Raf inhibitor.

In one preferred embodiment, the V600E B-Raf inhibitor is dabrafenib or vemurafenib.

In a most preferred embodiment, the kit-of-part composition comprises sirukumab or siltuximab and dabrafenib or vemurafenib.

In another embodiment, the kit-of-part composition comprises an anti-interleukin-6 receptor antibody and a chemotherapeutic agent.

In one particular embodiment, the anti-interleukin-6 receptor antibody tocilizumab or sarilumab

In another particular embodiment, the chemotherapeutic agent is a selective B-Raf inhibitor and preferably a V600E B-Raf inhibitor.

In a most preferred embodiment, the kit-of-part composition comprises tocilizumab or sarilumab and dabrafenib or vemurafenib.

Any inhibitor of STAT3 signaling pathway and chemotherapeutic agent of the invention as above described may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc. The pharmaceutical compositions of the invention can be formulated for a topical, oral, intranasal, intraocular, intravenous, intramuscular or subcutaneous administration and the like.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

To prepare pharmaceutical compositions, an effective amount of an inhibitor of STAT3 signaling pathway and a chemotherapeutic agent according to the invention may be dissolved or dispersed in a pharmaceutically acceptable carrier or aqueous medium.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, mixtures thereof and in oils. Under ordinary conditions of storage and use, these preparations contain a preservative to prevent the growth of microorganisms.

The inhibitors of STAT3 signaling pathway and chemotherapeutic agents according to the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution may be suitably buffered and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. In addition to the compounds formulated for parenteral administration, such as intravenous or intramuscular injection, other pharmaceutically acceptable forms include, e.g. tablets or other solids for oral administration; time release capsules; and any other form currently used.

The invention will be further illustrated by the following figures and examples. However, these examples and figures should not be interpreted in any way as limiting the scope of the present invention.

### FIGURES

FIG. 1: The secretome of senescent melanoma cells enhances the melanoma initiating cell population. (A and C) RNAs were harvested from 501mel cells exposed to the secretome of control melanoma cells transfected with control siRNA (Cont) or of melanoma cells rendered senescent by MITF silencing (SSMC) for 48 hrs and were assayed by qRT-PCR for transcripts indicated on the figure. Transcript levels are represented relative to those found in control cells as mean+SD. (B and D) Lysates from 501mel human melanoma cells exposed 48 hrs to the secretome of control melanoma cells (Cont) or of melanoma cells rendered senescent by exposure to temozolomide, 900 nM (SSMC-Temo) or fotemustine, 40 nM (SSMC-Fote), were analyzed by western blotting with MITF, ECAD, FN1, NANOG and OCT4 antibodies. ERIC2 was used as loading control. (E) Cells freshly isolated from human biopsies (Patient#1) exposed to the secretome of control melanoma cells (red line) or SSMC (black line) for 48 hrs, were analyzed for MITF content by flow cytometry. MITF intensity was plotted on abscissa and side scatter (SSC) on ordinates. An isotype-matched control antibody (green line) has been used as a negative control. (F) 501mel cells were labeled with 2 μM CFSE according to the manufacturer's protocol and grown in the conditioned media of secretome of control melanoma cells (red line) or SSMC (black line) for 72 hrs. Cells were next analyzed by flow cytometry for CFSE intensity. An estimation of the number of divisions (div) was determined according to CFSE intensity (0-4).

FIG. 2: Activation of the STAT3 signaling pathway by the secretome of senescent melanoma cells. (A) Human 501mel and WM9 melanoma cells lines and cells freshly isolated from human biopsies (Patient#1) were exposed 1 hr to the secretome of control (Cont) or senescent (SSMC) melanoma cells. Lysates were analyzed by western blot for the indicated antibodies. (B) ELISA test of IL6 level in control or senescent secretome. Data, represented as mean+SD, are significantly different ***p<0.001. (C) 501mel melanoma cells were treated with recombinant IL6 (20 ng/ml) or CCL2 (200 ng/ml) for the time indicated. Lysates were analyzed by western blot for the indicated antibodies. (D) 501mel cells in control condition or incubated with IL6 (20 ng/ml) for 48 hrs were analyzed for MITF content by flow cytometry. MITF intensity was plotted on abscissa and side scatter (SSC) on ordinates. (E) 501mel cells were left in control condition (red line) or incubated with IL6, 20 ng/ml for 72 hrs (black line). Cells were labeled with 2 M CFSE according to the manufacturer's protocol and next they were analyzed by flow cytometry for CFSE intensity. An estimation of the number of divisions (div) was determined according to CFSE intensity (0-4).

FIG. 3: STAT3 activation mediates the acquisition of the stemness phenotype in melanoma cells. (A-B) 501mel cells were infected with increasing concentrations of lentivirus encoding either the parental empty vector (EV) or vectors encoding either a constitutive active form (STAT3C) or a dominant negative form (STAT3DN) of STAT3. Western blotting assays were performed with the indicated antibodies. (C) QRT-PCR experiments on RNAs prepared from 501mel cells left in control condition, exposed to IL6 (20 ng/ml) or transfected with a STAT3 siRNA cells and treated with IL6. Transcript levels are represented relative to those found in control cells as mean+SD. (D) Western blotting assays of 501mel cells transfected with a control (siC) or STAT3 siRNA left untreated or treated with recombinant IL6. (E) FACS analysis of MITF intensity of cells treated as in D. MITF intensity was plotted on abscissa and side scatter (SSC) on ordinates.

FIG. 4: STAT3 activation is required for the acquisition of the tumorigenic phenotype of melanoma cells. (A-B) 501mel cells, transfected with a control (siC) or STAT3 siRNA, were left untreated or treated with recombinant IL6 (20 ng/ml) for 48 hrs and next were seeded on the filter of the upper compartment non coated (migration) or coated (invasion) with matrigel. DMEM with 7% serum were added to the lower compartment of Boyden chambers. Cells that had migrated to the underside of the upper compartment were stained 24 hrs later with crystal violet and the number of nuclei was counted using NIH-imageJ analysis software. Values represent mean+SD of three independent experiments, significantly different from the control ***p<0.001 and ** p<0.01. (C) 501mel cells, transfected with control (siC) or STAT3 siRNA were exposed to secretome of control melanoma cells (C), IL6 (20 ng/ml) or to the SSMC for 48 hrs and then subject to a cell viability assay. (D) 501mel melanoma cells treated as in (C) were xenografted in athymic nude mice. Values are expressed as mean±SEM.

FIG. 5: Model for melanoma chemoresistance. Chemotherapy treatment triggers cell death (1) and senescence (2) while sparing some cancer cells due to microenvironment or limited vascularisation (3). Senescent cells produce an inflammatory secretome composed of CCL2 and IL6 (4) that could enhance, through STAT3 activation, the survival and the acquisition of more mesenchymal and stemness properties of spared cancer cells (5), that in turn causes chemoresistance and relapses (6).

### EXAMPLE

### Material & Methods

Cell Cultures:

501mel, G361, WM9, MeWo and patient human melanoma cells (Patient#1) were grown in DMEM supplemented with 7% FBS at 37° C. in a humidified atmosphere containing 5% CO₂%. Cell isolation from human biopsies was previously described⁴⁶.

Antibodies and Reagents:

Anti-MITF (Ab80651) and anti-Nanog antibodies were from Abcam, anti-ERK2 (sc-1647 clone D-2) and anti-Oct4 antibodies were from Santa Cruz biotechnology. STAT3 (#9132), pTyr705STAT3 (#9131), pS473AKT (#9271), pThr202/Tyr204ERK1/2 (#9101) antibodies were from Cell Signaling Technology. Anti-fibronectin (#610077BD) and anti-E-cadherin (#610404BD) antibodies were from Transduction laboratories. Horseradish peroxidase-conjugated anti-rabbit or anti-mouse antibodies were from Dakopatts (Glostrup, Denmark). Recombinant human CCL2 and IL6 were from R&D Systems. Secondary alexa488 antibody, propidium iodide, DAPI, CellTrace CFSE and XTT cell proliferation kit were from Invitrogen.

Transient Transfection of siRNA:

Briefly, a single pulse of 50 nM of siRNA was administrated to the cells at 50% confluency by transfection with 5 μl Lipofectamine™ RNAiMAX in opti-MEM medium (Invitrogen, San Diego, Calif., USA). Control (siC) and MITF (siMi) siRNAs were previously described⁴⁷. STAT3 siRNA were purchased at Dharmacon Inc.

Migration and Invasion Assays:

Cell migration and invasion were assessed using a modified Boyden chamber assay with 8-μm pore filter inserts for 24-well plates (BD Bioscience). 501mel cells were seeded on the upper chamber of uncoated or matrigel-coated filters and DMEM 7% SVF placed into the lower chamber. Twenty-four hours later, cells adherent to the underside of the filters were fixed with 4% PFA, stained with 0.4% crystal violet and five random fields at ×20 magnification were counted. Results represent the average of triplicate samples from three independent experiments.

Western Blot Assays:

Western blots were carried out as previously described⁴⁸. Briefly, cell lysates (30 μg) were separated by SDS-PAGE, transferred on to a PVDF membrane and then exposed to the appropriate primary and HRP-linked secondary antibodies. Proteins were visualized with the ECL system (Amersham). The western blots shown are representative of at least 3 independent experiments.

mRNA Preparation, Real-Time/Quantitative PCR:

mRNA isolation was performed with Trizol (Invitrogen), according to standard procedure. QRT-PCR was carried out with SYBR® Green I and Multiscribe Reverse Transcriptase (Promega) and monitored by an ABI Prism 7900 Sequence Detection System (Applied Biosystems, Foster City, Calif.). Detection of SB34 gene was used to normalize the results. Primer sequences for each cDNA were designed using either Primer Express Software (Applied Biosystems) or qPrimer depot (http://primerdepot.nci.nih.gov) and are available upon request.

ELISA:

IL6 level in the secretome of 501mel melanoma cell lines was quantified by ELISA (R&D Systems). Results from two independent experiments were normalized to cell number and expressed as ng/ml/10⁶ cells.

Tumor Models:

Animal experiments were carried out in accordance with French law and were approved by a local institutional ethical committee. Animals were maintained in a temperature-controlled facility (22° C.) on a 12-hour light/dark cycle and were given free access to food (standard laboratory chow diet from UAR, Epinay-S/Orge, France).

Mice were randomly divided into 3 groups of 6 mice. 501mel melanoma cells were transfected with control (siC) or STAT3 (siSTAT3) siRNA and were exposed in vitro to the secretome of senescent melanoma cells (SSMC) or to recombinant CCL2 (200 ng/ml) or IL6 (20 ng/ml) for 48 hrs, washed twice with phosphate-buffered saline and then inoculated subcutaneously (4×10⁶ cells/mouse) into 6-week-old female immune-deficient Athymic Nude FOXN1^{nu} mice (Harlan Laboratory). The growth tumor curves were determined by measuring the tumor volume using the equation V=(L×W2)/2. Mice were killed by CO₂ inhalation and tumors were taken.

CFSE Labeling and FACS Analysis:

For the in vitro CFSE assay, cells were labeled with 2 μmol/l of CFSE according to the manufacturer's protocol (Invitrogen), then plated and treated with the secretome of control (SCMC) or senescent melanoma cells (SSMC) or recombinant IL6 (20 ng/ml). Cells were detached and analyzed by flow cytometry using MACSQuant (Miltenyi biotech). Cells were exposed to control (Cont) or senescent secretome (SSMC). Seventy-two hours later, cells were detached in phosphate-buffered saline/EDTA 2 mM and stained with DAPI to exclude dead cells. Fluorescence was measured by using the FL1/FL2 channels of a MACSQuant. Data were analyzed with MACSQuant software (Miltenyi biotech).

Cell Viability Test:

Cell viability was assessed using the cell proliferation kit II (XTT; Roche Molecular Biochemicals, Indianapolis, Ind.) according to the manufacturer's protocol. Cell viability, measured at 490 nm, is expressed as the percentage of the value of control cells.

Statistical Analysis:

Data are presented as averages±SD and were analyzed by student t-test using Microsoft Excel software. A p value of 0.05 (*p<0.05) or less (**p<0.01 and ***p<0.001) was interpreted as indicating statistical significance when comparing experimental and control groups.

Expression Profiling and Analysis:

Biotinylated cRNA was prepared with the Illumina TotalPrep RNA Amplification Kit (Ambion, Austin, Tex., USA). Labelled cRNA was hybridized to HumanHT-12 v4 BeadChip Arrays (Illumina Inc, San Diego, Calif., USA), and then washed and scanned according to standard Illumina protocols. Data were extracted in GenomeStudio (Illumina) using default analysis settings and no normalization method. Resulting data were imported into GeneSpring GX v11.5 (Agilent Technologies, Santa Clara, Calif., USA). Expression values were normalized using quantile normalization with default settings.

Results

The Secretome of Senescent Melanoma Cells Increases the Melanoma Initiating Cell Population:

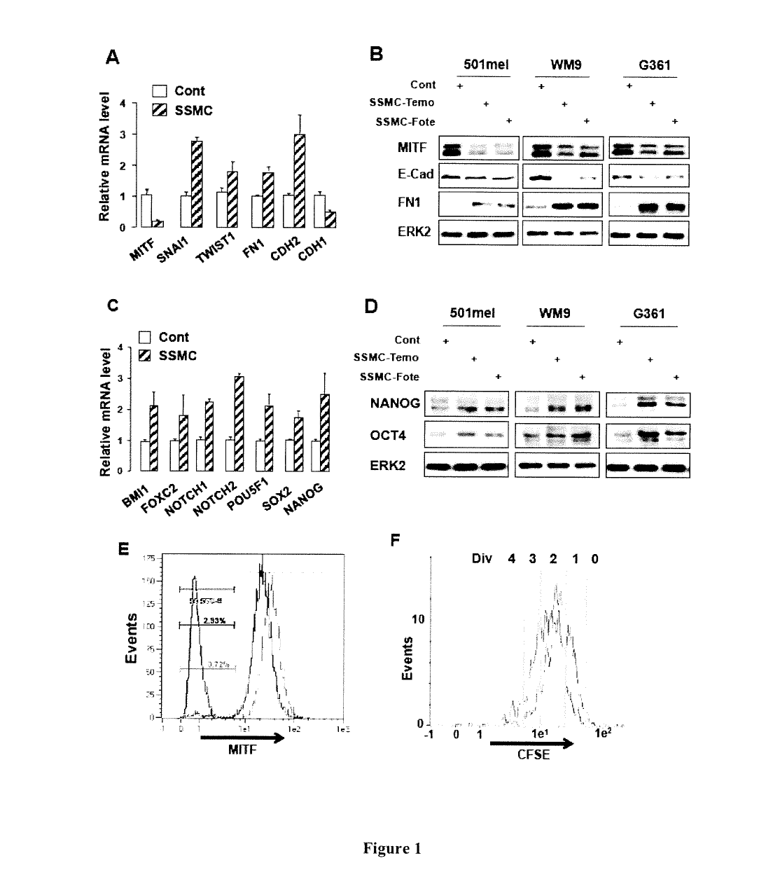

We have shown that exposure of naïve melanoma cells to the SSMC increases their motility and tumorigenic properties¹⁸. To explore the mechanism involved in these processes, we first analyzed the effect of this secretome on the expression of mesenchymal markers. QRT-PCR experiments showed that naïve 501mel melanoma cells exposed to the secretome of melanoma cells rendered senescent by MITF silencing exhibited an increased level of SNAIL1, TWIST1, Fibronectin1, N-Cadherin (CDH2) mRNAs whereas MITF and E-Cadherin (CDH1) transcripts were markedly decreased (FIG. 1A).

Similar effects were observed when senescence was induced by chemotherapy drugs used in first line treatment of melanoma. Indeed, three different melanoma cell lines exposed to the secretome collected from melanoma cells rendered senescent by temozolomide or fotemustine displayed increased expression of Fibronectin1 while exhibiting a decrease in E-Cadherin and MITF protein level (FIG. 1B, Supplementary FIG. S1A).

The loss of MITF and the gain of the mesenchymal phenotype were associated with an increase in stemness markers³. Therefore, we studied the effect of the secretome of melanoma cells rendered senescent by MITF silencing on these markers. QRT-PCR experiments showed that SSMC led to an increase in expression of several stemness markers including BMI1, FOXC2, NOTCH1/2, POU5F1 (OCT4), SOX2 and NANOG (FIG. 1C). We also observed increased expression of OCT4 and NANOG when naïve cells were exposed to the secretome of melanoma cells rendered senescent by the chemotherapy drugs (FIG. 1D).

These observations indicate that SSMC favors the acquisition of mesenchymal and stemness phenotypes by naive melanoma cells. These features are hallmarks of tumor-initiating cells, which were also characterized in melanoma by a low level of MITF expression and a transient slow growing rate^{3, 7}.

Flow cytometry analysis showed that exposure to SSMC increased the low-MITF population (70% to 300%) in 501mel, G361, MeWo and WM9 cell lines, which have different BRAF mutational status, and in melanoma cells isolated from patient skin metastasis expressing wild type BRAF, in which SSMC led to a 4-fold increase in the percentage of the low-MITF population (0.72 to 2.93%) (FIG. 1E). We also observed that SSMC decreased the growth of different melanoma cell lines, as illustrated by cell counting, but did not trigger cell death in 501mel and in other melanoma cells, in contrast to staurosporine used as a positive control. Then, using carboxyfluorescein diacetate succinimidyl ester (CFSE), a vital dye whose fluorescence intensity decreases by half at each cell division 23, we studied the division potential of melanoma cells exposed to SSMC. After CFSE loading, 501mel cells were cultured for 4 days with or without SSMC and analyzed by FACS (FIG. 1F). Under control conditions, most (80%) of 501mel cells underwent 2 to 3 divisions and a small percentage (8%) of cells underwent only 0/1 division (red line). When naïve melanoma cells were incubated with SSMC (black line) the percentage of the slow-growing cells (0/1 division) increased to 30%.

Therefore, naive melanoma cells, exposed to the conditioned medium of melanoma cells entering a program of senescence, experience a phenotypic switch that recapitulates the hallmarks of melanoma-initiating cells, i.e. increase in mesenchymal and stemness markers, augmentation of a low-MITF and slow-growing population.

Activation of the STAT3 Signaling Pathway by the Secretome of Melanoma Cells Undergoing Senescence:

To go deeper into the molecular mechanisms by which the senescent conditioned medium drives the reprogramming of melanoma cells, we assessed the effect of the SSMC on several signaling pathways that play critical roles in melanoma.

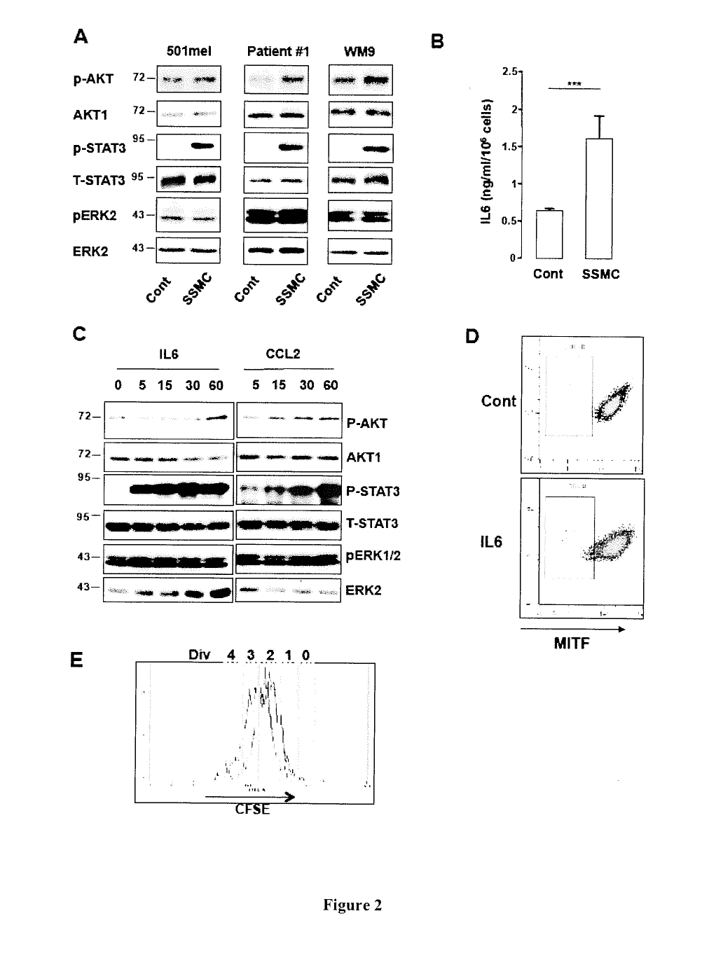

Western blot with phospho-specific antibodies directed against activated ERK, AKT or STAT3 indicated that melanoma cells of different genetic backgrounds exposed to SSMC displayed a strong activation of STAT3. No consistent activation of ERK and a weak AKT phosphorylation could be observed under the same conditions (FIG. 2A).

The STAT3 signaling pathway has been shown to be involved in the control of stemness and is activated by numerous cytokines present in the secretome of senescent melanoma, including CCL2¹⁸. Here, we extended these results and showed by ELISA that IL6 was also markedly increased in the SSMC (FIG. 2B).

Time course experiments showed that both CCL2 and IL6 induced a dramatic increase in STAT3 activation, as illustrated by its tyr705-phosphorylation (FIG. 2C). IL6 achieved a very rapid and robust STAT3 phosphorylation within 5 min, while the effect of CCL2 was more progressive and reached its maximum at 60 min Western blots also revealed that CCL2 and IL6 triggered no or very weak stimulation of AKT and ERK phosphorylation (FIG. 2C). Focusing on IL6, FACS analysis revealed that this cytokine increased the low-MITF population from 2 to 7% (FIG. 2D). IL6 also favored the apparition of the slow-growing population by increasing the percentage of cells that underwent 0/1 division (5.65 to 18.3%) (FIG. 2E). Therefore, IL6 is able to mimic the effects of the SSMC to favor the melanoma-initiating cell phenotype.

STAT3 Activation Mediates the Acquisition of the Stemness Phenotype Elicited by SSMC or by IL6:

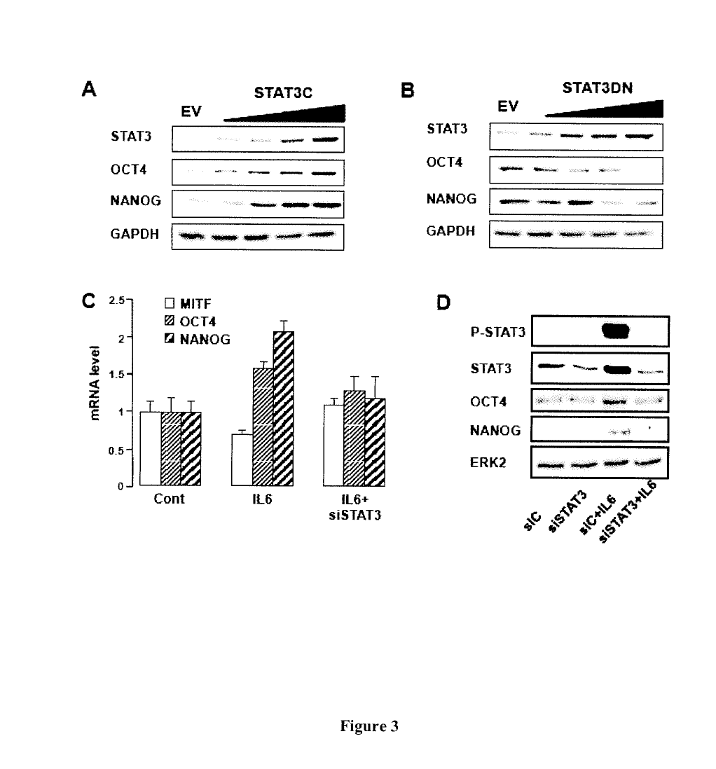

Overexpression of a constitutively active mutant of STAT3 (STAT3C) mimicked the effect of the SSMC or of IL6 on the stemness markers OCT4 and NANOG (FIG. 3A). Conversely, inhibition of STAT3 activity by overexpressing a dominant negative form (STAT3DN) promoted a decrease in OCT4 and NANOG protein level (FIG. 3B). Therefore, STAT3 activity appears to parallel the expression of NANOG and OCT4.

In agreement with the above observations, IL6 enhanced NANOG and OCT4 expression while it reduced the amount of MITF, at the mRNA and protein levels (FIG. 3C-D). STAT3 inhibition by siRNA prevented the effects of IL6 on OCT4 and NANOG expression. Additionally, flow-cytometry analysis revealed that the increase in the low-MITF population elicited by the SSMC (2% vs 6%) was inhibited by about 50% upon STAT3 silencing (6% vs 3.65%) (FIG. 3E). Hence, STAT3 activation is required for the acquisition of the melanoma-initiating cell properties induced by the SSMC or by IL6.

Next, we sought to identify the whole transcriptome modifications triggered by the SSMC. Towards this aim, two melanoma cell lines (501mel and WM9) and melanoma cells freshly isolated from a patient were transfected with STAT3 siRNA, or scrambled siRNA, then exposed or not to the SSMC. As shown by western blot in the three melanoma cell types, STAT3 siRNA efficiently reduced STAT3 expression and the SSMC activated STAT3 compared to the control conditioned medium. Furthermore, the reduced MITF expression mediated by SSMC was clearly abrogated in WM9 and patient#1 cells when STAT3 was knocked down. Expression array analysis highlighted a signature of 52 genes upregulated by the SSMC in the three melanoma cell types (Table 1). The regulation of all these genes was prevented by STAT3 inhibition, strengthening the key role of STAT3 in this process. It should be noted that Fibronectin1 (FN1), OCT4, NANOG and MITF do not belong to the 52-gene list. However, a careful analysis of the data revealed that most of these genes were below background detection limits or did not pass the statistical threshold.

Nevertheless, it should be noted that several known MITF target genes were weakly but significantly downregulated in all three melanoma cells, in accordance with the observations of a reduced MITF expression.

Re-assessment of the gene expression profile by qRT-PCR confirmed the markedly increased mRNA levels of AXL, ALDH1A3, CCL2, TNC, THBS2, DKK3 and TGFBI identified by the microarray experiments, also to a lesser extent that of FN1, OCT4, NANOG and the reduction of MITF. Ingenuity Pathway Analysis indicated that the major molecular and cellular functions associated with the genes enhanced by the SSMC were Cell movement, Cell-To-Cell Signaling and Interaction and Cellular Growth and Proliferation. Therefore, the SSMC induces a molecular reprogramming of melanoma cells toward a more motile and tumorigenic phenotype, mainly through STAT3 activation.

TABLE 1
List of the 52 genes regulated by the exposure to SSMC for 24 h, in 501Mel,
WM9 and melanoma cells from patient#1. Log ratio threshold >1. None of these genes is
regulated by SSMC in cells transfected with siRNA STAT3.

| Symbol | Log Ratio | p-value | Symbol | Log Ratio | p-value | Symbol | Log Ratio | p-value |
|---|---|---|---|---|---|---|---|---|
| DKK3 | 5.489 | 6.39E−03 | TMEM171 | 1.698 | 1.65E−02 | CHST3 | 1.32 | 1.46E−03 |
| SCRG1 | 4.378 | 1.66E−02 | CCL2 | 1.686 | 2.00E−02 | ARMCX2 | 1.27 | 1.20E−02 |
| TGFBI | 4.188 | 7.57E−04 | KLF9 | 1.674 | 2.55E−03 | SPOCK1 | 1.247 | 1.07E−02 |
| TIMP4 | 3.733 | 8.66E−03 | TMEM47 | 1.66 | 4.74E−03 | MYOF | 1.234 | 1.21E−02 |
| LOXL4 | 3.679 | 5.74E−03 | KCNMA1 | 1.569 | 2.50E−03 | STK32B | 1.224 | 3.22E−03 |
| ALDH1A3 | 3.495 | 4.17E−03 | A2M | 1.563 | 8.96E−04 | SYNM | 1.217 | 2.56E−03 |
| ST8SIA5 | 2.972 | 7.24E−03 | CDH13 | 1.533 | 2.08E−03 | CA12 | 1.187 | 2.83E−03 |
| TIMP3 | 2.796 | 2.36E−03 | PDGFRA | 1.514 | 1.08E−03 | TNFRSF6B | 1.184 | 1.47E−02 |
| PXDN | 2.767 | 1.13E−03 | IL1RAPL1 | 1.502 | 1.00E−02 | LAMA4 | 1.15 | 2.02E−03 |
| SFRP1 | 2.45 | 1.99E−02 | PASD1 | 1.448 | 1.75E−02 | PRSS23 | 1.145 | 1.20E−02 |
| HTATIP2 | 2.225 | 6.54E−03 | SDC4 | 1.429 | 3.78E−03 | GBP2 | 1.087 | 3.37E−03 |
| PCOLCE | 2.134 | 1.05E−02 | SLFN11 | 1.348 | 1.63E−03 | HS3ST3A1 | 1.083 | 5.72E−03 |
| BGN | 2.126 | 1.20E−02 | VAT1L | 1.323 | 8.13E−03 | TNS3 | 1.064 | 5.96E−03 |
| IL1B | 2.096 | 1.55E−02 | CHST3 | 1.32 | 1.46E−03 | ITIH6 | 1.059 | 4.02E−03 |
| AXL | 2.072 | 4.65E−03 | PDGFRA | 1.514 | 1.08E−03 | SRPX | 1.057 | 1.29E−03 |
| PMEPA1 | 1.889 | 4.80E−03 | IL1RAPL1 | 1.502 | 1.00E−02 | EFEMP2 | 1.048 | 1.36E−02 |
| FAM129A | 1.815 | 2.44E−04 | PASD1 | 1.448 | 1.75E−02 | CRMP1 | 1.028 | 3.22E−03 |
| COL8A1 | 1.79 | 3.02E−03 | SDC4 | 1.429 | 3.78E−03 | GNG2 | 1.025 | 4.23E−03 |
| RGL1 | 1.73 | 6.13E−03 | SLFN11 | 1.348 | 1.63E−03 | SLC38A1 | 1.024 | 5.70E−03 |
| EIF1AY | 1.724 | 1.30E−02 | VAT1L | 1.323 | 8.13E−03 | | | |

STAT3 Activation Mediates the Acquisition of Tumorigenic Phenotype Elicited by SSMC or by IL6 and is Required for Melanoma Growth In Vivo:

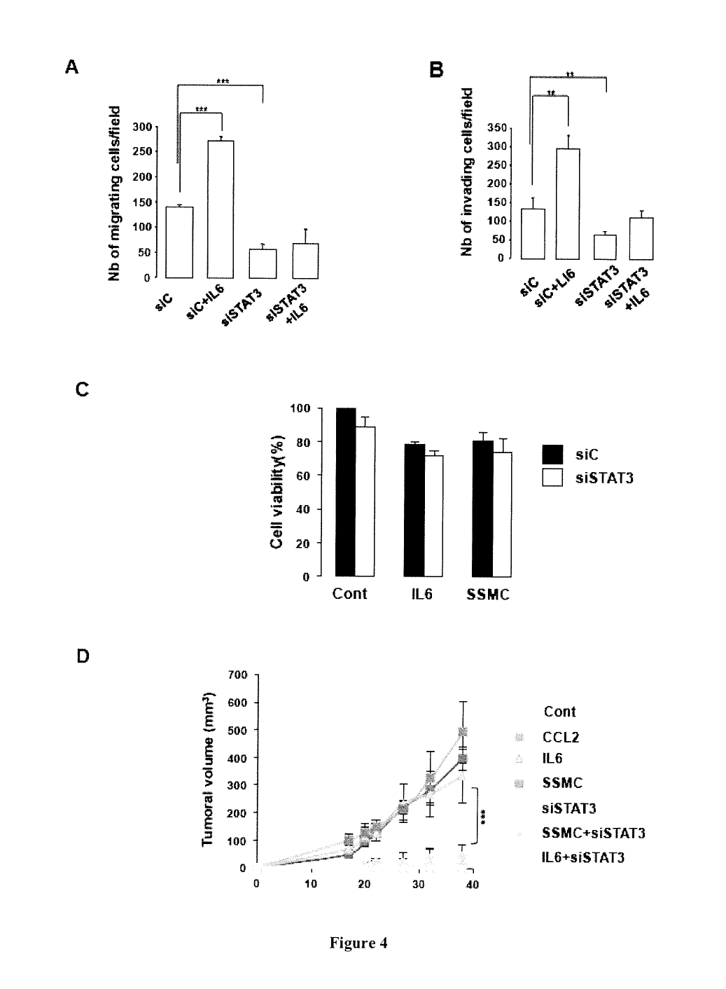

Finally, we evaluated the effect of STAT3 silencing on the biological behavior of melanoma cells. Boyden chamber experiments demonstrated that STAT3 siRNA decreased both basal and IL6 induced-migration and invasion (FIG. 4A-B). However, STAT3 siRNA did not affect significantly cell growth or viability (FIG. 4C). To determine the effect of the secretome in vivo, we used 501mel human melanoma cells that do not grow as xenografts in athymic nude mice. Cells exposed in vitro to secretome of control melanoma cells or cells transfected with STAT3 siRNA before transplantation did not form tumors. However, 501mel exposed to SSMC, CCL2 or IL6 gave rise to tumors (FIG. 4D). Importantly, the pro-tumorigenic effects of SSMC or of IL6 were completely abrogated when cells were knocked down for STAT3, thereby demonstrating the key role of STAT3 activation in the acquisition of the tumorigenic potential by melanoma cells.

### Discussion

Heterogeneity and plasticity are the two biological phenomena that might be responsible for the remarkable resistance of melanoma to the current therapeutic armamentarium. Both phenomena can be explain by the concept of melanoma initiating cells which are thought to derive from the phenotypic switch of more differentiated melanoma cells²⁴. It has been shown that stimuli such as hedgehog²⁵ or hypoxia⁹ can increase the MIC population, favoring thereby tumorigenicity. We previously reported that chemotherapy drugs, used in melanoma treatment, entail a senescence-like phenotype in melanoma cells that is associated with the production of an inflammatory secretome (SSMC) endowed with pro-tumorigenic properties¹⁸. Therefore, we hypothesized that SSMC might favor melanoma tumorigenicity also by increasing MIC population.

Here, we show that the SSMC and IL6, one of its components, enhance the expression of mesenchymal and stemness markers, that pairs with an increase in the MIC population defined by a slow-growing rate and a low-MITF expression. These observations are in agreement, with several reports demonstrating that IL6, which belongs to the LIF family, favors the transition toward cancer stem cells in breast and prostate cancers²⁶, and increases mesenchymal transition of melanoma cells and melanoma tumor development²⁷. Further, melanoma development is delayed in IL6 deficient mice²⁸.

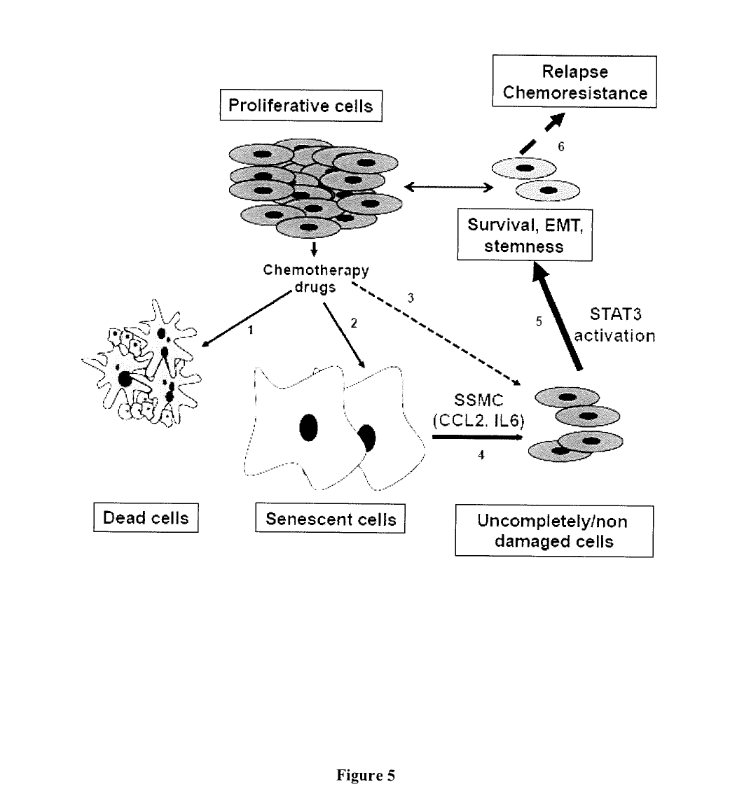

Study of the signaling pathways engaged by exposure to the SSMC showed no activation of ERK, a weak activation of AKT and a robust activation of STAT3. The Signal Transducer and Activator of Transcription-3 (STAT3) is a member of the STAT family that relays extracellular signals initiated by cytokines and growth factors from the cytoplasm to the nucleus²⁹. The constitutive activation of STAT3 is frequently detected in human cancer, including melanoma, and is associated with poor clinicopathological features and prognosis^{30,31}. Recently, STAT3 activation has been associated with tumor initiating cell phenotype of liver, colon and glial cancer cells³²⁻³⁴. Further, the activation of STAT3 has been also involved in the acquisition/maintenance of the pluripotency by controlling the expression of KLF4 and NANOG³⁵. Our results demonstrate that CCL2 and IL6, which are present in the SSMC, also promote an activation of STAT3 and that STAT3 silencing prevents the acquisition of the mesenchymal, stemness and melanoma-initiating phenotypes. Collectively these observations indicate that STAT3 is a key player in the transition between melanoma initiating cells and their more differentiated progeny (FIG. 5).

Therefore, we sought to identify the repertoire of genes regulated by the SSMC that are under the control of STAT3. Transcriptomic studies and Ingenuity Pathway Analysis revealed that SSMC increases the expression of a set of genes related to Cell movement, Cell-To-Cell Signaling and Interaction and Cellular Growth and Proliferation. Among these genes, 15 are both up-regulated in melanoma cell lines with invasive phenotype³⁶ and downregulated in A375 melanoma cells over-expressing MITF³⁷. Therefore, the transcriptomic profile matches perfectly with the acquisition of a more invasive phenotype and melanoma-initiating cell properties. The inhibition of STAT3 by siRNA completely prevented the regulation of all these genes, reinforcing the notion that STAT3 is a critical mediator of melanoma-initiating features and melanoma aggressiveness.

Among the direct or indirect STAT3 targets, upregulated by SSMC; ALDH1A3 was recently associated with melanoma-initiating properties^{25, 38}, even though not all reports agree with this hypothesis³⁹; tenascin (TNC), an extracellular matrix protein is an important component of stem cell niches⁴⁰ that favors evasion of tumor cells, including melanoma⁴¹, from conventional therapy and might therefore participate in the acquisition of the MIC phenotype. Finally, the upregulation of AXL by SSMC is of particular interest, because AXL belongs to the TAM (Tyro3, AXL, MER) family of receptor tyrosine kinase involved in various cancers, including melanoma⁴². AXL was thought to play a key role in the acquisition of the resistance to EGFR inhibitor in lung cancer⁴³. In melanoma, AXL is activated and preferentially expressed in melanoma lacking MITF⁴⁴. In agreement with our data, AXL favors melanoma migration.

How STAT3 regulates these genes remains to be elucidated. Of course STAT3 can directly bind to the promoter of the up-regulated genes to control their expression. However, the transient activation of STAT3 during in vitro exposure of melanoma cells to SSMC is sufficient to increase the tumorigenic potential and favors the subsequent tumor development, indicating that the consequences of STAT3 activation continue for several weeks and cell divisions, to allow tumor development. This effect persisting despite the cessation of the initial stimuli might also be the consequence of an epigenetic regulation. This hypothesis is in agreement with a recent report demonstrating that STAT3 signaling promotes somatic cell reprogramming by epigenetic regulation⁴⁵ and may provide a link between the inflammatory response, epigenetic remodeling and cancer development.

Finally, the inhibition of STAT3 expression by siRNA abrogates the acquisition of the tumorigenic properties evoked by the in vitro exposure of melanoma cells to SSMC and completely prevents the development of xenografts. This observation strengthens the pivotal role of STAT3 in melanoma development and provides a rational for evaluation of STAT3 inhibitors in melanoma treatment, alone or in combination with existing therapies.

### REFERENCES

Throughout this application, various references describe the state of the art to which this invention pertains. The disclosures of these references are hereby incorporated by reference into the present disclosure.
1. Orgaz, J. L. & Sanz-Moreno, V. Emerging molecular targets in melanoma invasion and metastasis. Pigment cell & melanoma research (2012).
2. Tawbi, H. A. & Buch, S. C. Chemotherapy resistance abrogation in metastatic melanoma. Clinical advances in hematology & oncology: H & O8, 259-266 (2010).
3. Cheli, Y. et al. Mitf is the key molecular switch between mouse or human melanoma initiating cells and their differentiated progeny. Oncogene 30, 2307-2318 (2011).
4. Boiko, A. D. et al. Human melanoma-initiating cells express neural crest nerve growth factor receptor C D271. Nature 466, 133-137 (2010).
5. Fang, D. et al. A tumorigenic subpopulation with stem cell properties in melanomas. Cancer research 65, 9328-9337 (2005).
6. Pinner, S. et al. Intravital imaging reveals transient changes in pigment production and Brn2 expression during metastatic melanoma dissemination. Cancer research 69, 7969-7977 (2009).
7. Roesch, A. et al. A temporarily distinct subpopulation of slow-cycling melanoma cells is required for continuous tumor growth. Cell 141, 583-594 (2010).
8. Schatton, T. et al. Identification of cells initiating human melanomas. Nature 451, 345-349 (2008).
9. Cheli, Y. et al. Hypoxia and MITF control metastatic behaviour in mouse and human melanoma cells. Oncogene 31, 2461-2470 (2012).
10. Harbst, K. et al. Molecular profiling reveals low- and high-grade forms of primary melanoma. Clinical cancer research: an official journal of the American Association for Cancer Research 18, 4026-4036 (2012).
11. Hoek, K. S. et al. In vivo switching of human melanoma cells between proliferative and invasive states. Cancer research 68, 650-656 (2008).
12. Steingrimsson, E., Copeland, N. G. & Jenkins, N. A. Melanocytes and the microphthalmia transcription factor network. Annu Rev Genet 38, 365-411 (2004).
13. Giuliano, S., Ohanna, M., Ballotti, R. & Bertolotto, C. Advances in melanoma senescence and potential clinical application. Pigment cell & melanoma research 24, 295-308 (2010).
14. Mhaidat, N. M. et al. Temozolomide induces senescence but not apoptosis in human melanoma cells. Br J Cancer 97, 1225-1233 (2007).
15. Strub, T. et al. Essential role of microphthalmia transcription factor for DNA replication, mitosis and genomic stability in melanoma. Oncogene 30, 2319-2332 (2011).
16. Coppe, J. P., Desprez, P. Y., Krtolica, A. & Campisi, J. The senescence-associated secretory phenotype: the dark side of tumor suppression. Annu Rev Pathol 5, 99-118 (2010).
17. Kuilman, T. & Peeper, D. S. Senescence-messaging secretome: SMS-ing cellular stress. Nature reviews. Cancer 9, 81-94 (2009).
18. Ohanna, M. et al. Senescent cells develop a PARP-1 and nuclear factor-{kappa}B-associated secretome (PNAS). Genes & development 25, 1245-1261 (2011).
19. Acosta, J. C. et al. Chemokine signaling via the CXCR2 receptor reinforces senescence. Cell 133, 1006-1018 (2008).
20. Kuilman, T. et al. Oncogene-induced senescence relayed by an interleukin-dependent inflammatory network. Cell 133, 1019-1031 (2008).
21. Canino, C. et al. SASP mediates chemoresistance and tumor-initiating-activity of mesothelioma cells. Oncogene (2011).
22. Laberge, R. M., Awad, P., Campisi, J. & Desprez, P. Y. Epithelial-Mesenchymal Transition Induced by Senescent Fibroblasts. Cancer Microenviron (2011).
23. Lyons, A. B., Hasbold, J. & Hodgkin, P. D. Flow cytometric analysis of cell division history using dilution of carboxyfluorescein diacetate succinimidyl ester, a stably integrated fluorescent probe. Methods in cell biology 63, 375-398 (2001).
24. Hoek, K. S. & Goding, C. R. Cancer stem cells versus phenotype-switching in melanoma. Pigment cell & melanoma research 23, 746-759 (2010).
25. Santini, R. et al. HEDGEHOG-GLI Signaling Drives Self-Renewal and Tumorigenicity of Human Melanoma-Initiating Cells. Stem Cells 30, 1808-1818 (2012).
26. Iliopoulos, D., Hirsch, H. A., Wang, G. & Struhl, K. Inducible formation of breast cancer stem cells and their dynamic equilibrium with non-stem cancer cells via IL6 secretion. Proceedings of the National Academy of Sciences of the United States of America 108, 1397-1402 (2011).
27. Kushiro, K. & Nunez, N. P. Ob/ob serum promotes a mesenchymal cell phenotype in Bl6BL6 melanoma cells. Clin Exp Metastasis 28, 877-886 (2011).
28. von Felbert, V. et al. Interleukin-6 gene ablation in a transgenic mouse model of malignant skin melanoma. The American journal of pathology 166, 831-841 (2005).
29. Frank, D. A. STAT3 as a central mediator of neoplastic cellular transformation. Cancer Lett 251, 199-210 (2007).
30. Wu, Z. S., Cheng, X. W., Wang, X. N. & Song, N. J. Prognostic significance of phosphorylated signal transducer and activator of transcription 3 and suppressor of cytokine signaling 3 expression in human cutaneous melanoma. Melanoma Res 21, 483-490 (2011).
31. Yu, H., Pardoll, D. & Jove, R. STATs in cancer inflammation and immunity: a leading role for STAT3. Nature reviews. Cancer 9, 798-809 (2009).
32. Lee, T. K. et al. CD24(+) liver tumor-initiating cells drive self-renewal and tumor initiation through STAT3-mediated NANOG regulation. Cell stem cell 9, 50-63 (2011).
33. Lin, L. et al. STAT3 is necessary for proliferation and survival in colon cancer-initiating cells. Cancer research 71, 7226-7237 (2011).
34. Villalva, C. et al. STAT3 is essential for the maintenance of neurosphere-initiating tumor cells in patients with glioblastomas: a potential for targeted therapy? International journal of cancer. Journal international du cancer 128, 826-838 (2011).
35. Hasegawa, Y. et al. CC chemokine ligand 2 and leukemia inhibitory factor cooperatively promote pluripotency in mouse induced pluripotent cells. Stem Cells 29, 1196-1205 (2011).
36. Widmer, D. S. et al. Systematic classification of melanoma cells by phenotype-specific gene expression mapping. Pigment cell & melanoma research 25, 343-353 (2012).
37. Bertolotto, C. et al. A SUMOylation-defective MITF germline mutation predisposes to melanoma and renal carcinoma. Nature 480, 94-98 (2011).
38. Luo, Y. et al. ALDH1A isozymes are markers of human melanoma stem cells and potential therapeutic targets. Stem Cells 30, 2100-2113 (2012).
39. Prasmickaite, L. et al. Aldehyde dehydrogenase (ALDH) activity does not select for cells with enhanced aggressive properties in malignant melanoma. PloS one 5, e10731 (2010).
40. Brellier, F. et al. Tenascin-C triggers fibrin accumulation by downregulation of tissue plasminogen activator. FEBS letters 585, 913-920 (2011).
41. Fukunaga-Kalabis, M. et al. Tenascin-C promotes melanoma progression by maintaining the ABCB5-positive side population. Oncogene 29, 6115-6124 (2010).
42. Linger, R. M., Keating, A. K., Earp, H. S. & Graham, D. K. TAM receptor tyrosine kinases: biologic functions, signaling, and potential therapeutic targeting in human cancer. Advances in cancer research 100, 35-83 (2008).
43. Byers, L. A. et al. An epithelial-mesenchymal transition (EMT) gene signature predicts resistance to EGFR and PI3K inhibitors and identifies Axl as a therapeutic target for overcoming EGFR inhibitor resistance. Clinical cancer research an official journal of the American Association for Cancer Research (2012).
44. Sensi, M. et al. Human cutaneous melanomas lacking MITF and melanocyte differentiation antigens express a functional Axl receptor kinase. The Journal of investigative dermatology 131, 2448-2457 (2011).
45. Tang, Y. et al. Jak/Stat3 signaling promotes somatic cell reprogramming by epigenetic regulation. Stem Cells 30, 2645-2656 (2012).
46. Giuliano, S. et al. Microphthalmia-associated transcription factor controls the DNA damage response and a lineage-specific senescence program in melanomas. Cancer research 70, 3813-3822 (2010).
47. Larribere, L. et al. The cleavage of microphthalmia-associated transcription factor, MITF, by caspases plays an essential role in melanocyte and melanoma cell apoptosis. Genes & development 19, 1980-1985 (2005).
48. Hilmi, C. et al. IGF1 promotes resistance to apoptosis in melanoma cells through an increased expression of BCL2, BCL-X(L), and survivin. The Journal of investigative dermatology 128, 1499-1505 (2008)
49. Haferkamp S, Borst A, Adam C, Becker T M, Motschenbacher S, Windhövel S, Hufnagel A L, Houben R, Meierjohann S. Vemurafenib induces senescence features in melanoma cells. J Invest Dermatol. 2013 June; 133(6):1601-9.

1. A method for inhibiting the tumorigenicity of senescent cancer cells induced by a chemotherapeutic agent in a patient in need thereof, comprising
administering to said patient (i) the chemotherapeutic agent and (ii) an inhibitor of the signal transducer and activator of transcription 3 (STAT3) signaling pathway.

2. The method of claim 1, wherein said senescent cancer cells are senescent melanoma cells.

3. The method of claim 1, wherein said inhibitor of STAT3 signaling pathway is a STAT3 inhibitor or an IL-6 antagonist.

4. The method of claim 3, wherein said STAT3 inhibitor is an inhibitor of STAT3 gene expression.

5. The method of claim 3, wherein said IL-6 antagonist is an anti-IL-6 antibody or an anti-IL-6 receptor (IL-6R) antibody.

6. The method of claim 5, wherein said anti-IL-6 antibody is sirukumab or siltuximab.

7. The method of claim 5, wherein said anti-IL-6R antibody is tocilizumab or sarilumab.

8. The method of claim 1, wherein said chemotherapeutic agent is an anti-melanoma drug.

9. The method of claim 1, wherein said chemotherapeutic agent and said inhibitor of the STAT3 signaling pathway are administered simultaneously or sequentially.

10. A method for inhibiting the conversion of non-stem cancer cells (non-CSCs) into cancer stem cells (CSCs) induced by a chemotherapeutic agent comprising,
administering to said subject (i) the chemotherapeutic agent and (ii) an inhibitor of the signal transducer and activator of transcription 3 (STAT3) signaling pathway.

11. The method of claim 10, wherein said non-CSCs are naive melanoma cells and said CSCs are melanoma initiating cells (MICs).

12. A pharmaceutical composition for inhibiting the conversion of non-stem cancer cells (non-CSCs) into cancer stem cells (CSCs) induced by a chemotherapeutic agent comprising
(i) the chemotherapeutic agent,
(ii) an inhibitor of STAT3 signaling pathway, and
(iii) a physiologically acceptable carrier.

13. The pharmaceutical composition of claim 12, wherein said inhibitor of STAT3 signaling pathway is a STAT3 inhibitor or an IL-6 antagonist.

14. The pharmaceutical composition of claim 13, wherein said STAT3 inhibitor is an inhibitor of STAT3 gene expression.

15. The pharmaceutical composition of claim 13, wherein said IL-6 antagonist is an anti-IL-6 antibody or an anti-IL-6 receptor (IL-6R) antibody.

16. The pharmaceutical composition of claim 15, wherein said anti-IL-6 antibody is sirukumab or siltuximab.

17. The pharmaceutical composition of claim 15, wherein said anti-IL-6R antibody is tocilizumab or sarilumab.

18. The pharmaceutical composition of claim 12, wherein said chemotherapeutic agent is an anti-melanoma drug.

19. A pharmaceutical composition or a kit-of-part composition comprising
i) an anti-IL-6 antibody or an anti-IL-6R antibody, and
ii) a V600E B-Raf inhibitor.

20. The pharmaceutical composition or the kit-of-part composition according to claim 19, wherein the anti-IL-6 antibody is sirukumab or siltuximab and the V600E B-Raf inhibitor is dafrafenib or vemurafenib.

21. The pharmaceutical composition or the kit-of-part composition according to claim 19, wherein the anti-IL-6R antibody is tocilizumab or sarilumab and the V600E B-Raf inhibitor is is dafrafenib or vemurafenib.
